(19) United States
(12) Patent Application Publication
(10) Pub. No.: US 2013/0183394 A1
(43) Pub. Date: Jul. 18, 2013

(54) COSMETIC OR DERMATOLOGICAL COMPOSITION FOR TOPICAL APPLICATION

(71) Applicant: Wolfgang Wuttke, Bovenden (DE)

(72) Inventor: Wolfgang Wuttke, Bovenden (DE)

(73) Assignee: BIONORICA AG, Neumarkt (DE)

(21) Appl. No.: 12/678,754

(22) PCT Filed: Sep. 19, 2008
(86) PCT No.: PCT/DE2008/001561

§ 371 Date: Jun. 4, 2010

(30) Foreign Application Priority Data
Sep. 19, 2007 (DE) 10 2007 044 916.1

(51) Int. Cl. A61K 36/71; A61K 36/28; A61K 36/185; A61K 36/87; A61K 36/48

(52) U.S. Cl. CPC A61K 36/71; A61K 36/87; A61K 36/48; A61K 36/185; A61K 36/28
USPC 424/757; 424/725; 424/766

(57) ABSTRACT

The invention relates to a cosmetic or dermatological composition, preferably purely plant-based, for topical use, containing Cimicifuga as the primary natural active ingredient, and to the use thereof for the prophylaxis and the treatment of skin diseases, in particular acne, seborrhea, atopic eczema (neurodermatitis), hirsutism, psoriasis, and dry/atopic skin of humans.

The invention relates to a cosmetic or dermatological composition, which is preferably purely plant-based, for topical use, comprising Cimicifuga as the primary natural active ingredient, and to the use thereof for the prophylaxis and the treatment of skin diseases, in particular acne, seborrhea, atopic eczema (neurodermatitis), hirsutism, psoriasis, and dry/atopic skin of humans.

A modern cosmetic or dermatological composition, which preferably is purely plant-based, for topical use is subject to high demands.

As the largest organ of humans, the skin performs a variety of vital functions. For example, it offers protection from the cold, heat, from radiation, the effects of chemical substances, and pathogens. When the skin no longer sufficiently performs this barrier function, local irritations or even symptoms affecting the entire body may develop.

As the barrier organ of the human organism, the skin, primarily the epidermis, is particularly exposed to external influences. This barrier function is maintained by skin lipids, among other things. These epidermal lipids, such as glycosphingolipids, ceramides, sterols and sterol esters, fatty acids, triglycerides, n-alkanes, or different polar lipids, are released during the ceratinization process.

In an optimal state of the skin, there is a balance between skin lipids and skin moisture. This balance plays a role in determining key properties of the skin, such as the penetration ability, hydration ability, elasticity, regeneration ability, or resistance to environmental influences and a wide variety of toxic agents.

Thus, superior importance is attached to the skin lipids, in particular the surface lipids.

The outer lipid film of the skin is formed by an emulsion having a very complex composition of different lipids and the secretion of the perspiratory glands, such as urea, fatty acids, inorganic salts, and water. The constituents of the oil phase are predominantly secretions from the sebaceous glands containing, among other things, squalene, cholesterol and cholesterol esters, wax esters, triglycerides, and free fatty acids.

In the prior art, in US 2003007939, black cohosh is disclosed as a minor constituent in a pharmaceutical composition for the treatment of skin diseases. CN 1196037 discloses the oral application of Cimicifuga in the form of tablets for treating acne. The disadvantage, however, is the low efficacy by way of oral application. DE 3641220 discloses the use of Cimicifuga, in addition to other constituents, for the treatment of skin diseases, however Cimicifuga is administered only in homeopathic doses.

In CN 1120952, a multi-component mixture for the treatment of acne is disclosed, which also includes Cimicifuga. Cimicifuga, however, is not the primary active ingredient, but only a secondary constituent. Furthermore, the primary constituent of this mixture is present in a Vaseline cream having disadvantageous properties for the skin. While Vaseline is a cost-effective base material, it results in toxic effects in the skin.

The applicant has already proven that Cimicifuga can inhibit 5-alpha-reductase (EP 1064009B1, EP1427430B1). Cimicifuga extracts are therefore likewise suited for the therapy of prostate cancer. 5-alpha-reductase is an enzyme which metabolizes testosterone circulating in the serum to 5-alpha dihydrotestosterone (5-alpha-DHT) locally in the prostate gland and in the skin appendages. This is the androgen that in fact is effective. In the skin (excluding the scalp), however, 5-alpha-DHT stimulates sebum production and hair growth.

It is the object of the present invention to provide a cosmetic or dermatological composition for topical use, which, in addition to providing skin care, is suited to treat skin diseases, particularly acne, psoriasis, seborrhea, atopic eczema (neurodermatitis), and dry/atopic skin of humans and to achieve an accordingly improved cosmetic or dermatological composition for topical use.

The object is achieved by a cosmetic or dermatological preparation for topical use, comprising 1.0 to 50% by weight Cimicifuga as the primary active ingredient. In further embodiments, preferably 2.0 to 30% by weight, particularly preferably 2.5 to 20% by weight Cimicifuga, and particularly preferably 2.5 to 10% by weight, is used in the preparation according to the invention. These embodiments can be used for conventional skin care and, at the same time, for treating skin diseases and dry/atopic skin in an efficient and complex manner and achieve excellent results.

The selected ranges of the primary active ingredient Cimicifuga according to the invention particularly advantageously allow an optimal effect on 5-alpha-reductase within the context of topical use. The composition according to the invention can be applied to the skin preferably in the form of a skin cream or emulsion, ointment (paste, cream, gel) or lotion.

Within the scope of the present invention, “Cimicifuga” is preferably Cimicifuga racemosa (black cohosh from the family Ranunculaceae), in particular an extract or dry extract, and in particular from the rootstock (Rhizoma cimicifugae racemosae, refer to EP1427430B1).

“Primary active ingredient” shall mean that, in addition to other active ingredients, the active ingredient Cimicifuga is present in a predominant quantity in the composition according to the invention.

Accordingly, secondary active ingredients may be present in an active ingredient combination, in addition to the primary active ingredient Cimicifuga. Such active ingredients are preferably extracts of soy, red clover, hops (Humulus, particularly Humulus lupulus), grape skins and calendula, in particular such extracts containing a high percentage of isoflavones.

Following percutaneous application, these secondary active ingredients promote the action of Cimicifuga.

In a particularly preferred embodiment, the composition according to the invention is purely plant-based.

Such an embodiment can, for example, advantageously comprise unsaponifiable constituents of at least one further vegetable oil, and in particular no mineral oils or fats from residues of crude oil distillation (such as Vaseline and many others).

Within the scope of the present invention, “unsaponifiable constituents of at least one further vegetable oil” shall encompass the collectivity of liposoluble and water-insoluble organic constituents of at least one plant which cannot be converted into salts by way of a saponification reaction (such as according to DIN 53900, technical regulation, Germany). Vegetable fats comprise at least 0.1 to 5% by weight unsaponifiable constituents. The compositions of these unsaponifiable constituents are not exhaustive: aromatic compounds, terpenes, tocopherols, carotenes, C-16 to C-22 hydrocarbons, terpene alcohols, C-16 to C-22 alcohols, and different sterols, such as cholesterol, alpha-sitosterol, stigmasterol, campesterol, brassicasterol, and phystosterols. These constituents shall not be understood as active ingredients, but instead support the topical use of the composition according to the invention.

Within the scope of the present invention, the term “atopic eczema” (also: atopic dermatitis or ‘neurodermatitis’) shall be understood as an increasingly frequent skin disease in the form of flare-ups, affecting especially adolescents. The skin of a person afflicted by neurodermatitis is highly sensitive during the acute phase and can manifest as allergic reactions to mental and physical stress factors and a number of individually different environmental factors and toxins. During the acute phase, the skin is inflamed and the person affected suffers in particular from agonizing itchiness. From what is known today, neurodermatitis has no underlying illness of the inner organs, but relates inflammatory free radical-producing processes of the outer skin layers, so that the external application of ointments, creams, or gels can positively influence the disease.

In a further embodiment, the cosmetic or dermatological preparation for topical use according to the invention can take on the form of an ointment, cream, gel, lotion, paste, or preferably emulsion. Anhydrous systems are likewise possible.

In general, emulsions are heterogeneous systems comprising two fluids which cannot be mixed with each other, or only to a very limited extent, and typically are referred to as phases. In an emulsion, one of the two fluids is dispersed into the other fluid in the form of very fine droplets. If the two fluids are water and oil, and oil droplets are present finely distributed in the water, it is an oil-in-water emulsion (O/W emulsion). The basic nature of an O/W emulsion is influenced by the water. A water-in-oil emulsion (N/O emulsion) involves the opposite principle, wherein the basic nature is determined by the oil. Furthermore, multiple systems such as water-in-oil-in-water emulsions (W/O/W emulsion) and oil-in-water-in-oil emulsions (O/W/O emulsion) are known. All emulsions mentioned here are suitable according to the invention.

The anhydrous systems suitable according to the invention include pure oil preparations, such as skin oils. In addition, possible pastes containing the preparation according to the invention are characterized in that they comprise the same or similar constituents as an emulsion, but are substantially anhydrous. Within the scope of the present invention, the terms oil phase and lipid phase are used as synonyms. In a further preferred embodiment, the preparation according to the invention may comprise an emulsifier as an additional constituent. In a particularly preferred embodiment, this emulsifier can be an O/W emulsifier.

Emulsifiers can advantageously be selected from the group of non-ionic, anionic, cationic, or amphoteric emulsifiers.

The non-ionic emulsifiers used can be different emulsifiers from the groups consisting of partial fatty acid esters, fatty alcohols, sterols, polyethylene glycols, such as ethoxylated fatty acids, ethoxylated fatty alcohols, and ethoxylated sorbitan esters, sugar emulsifiers, polyglycerin emulsifiers, or silicon emulsifiers.

The anionic emulsifiers used can be different emulsifiers from the groups consisting of soaps, such as sodium stearate, fatty alcohol sulfates, mono-, and tri-alkyl phosphoric acid esters and the ethoxylates thereof, fatty acid lactate esters, fatty acid citrate esters, or fatty acid citroglycerin esters.

Cationic emulsifiers can be, for example, quaternary ammonium compounds having a long-chain aliphatic group, such as distearyldimonium chloride.

Amphoteric emulsifiers can include different emulsifiers from the groups consisting of alkylamininoalkane carboxylic acids, betaines, sulfobetaines, or irnidazoline derivatives.

According to the invention, naturally occurring emulsifiers are preferred, which include, for example, bee's wax, wool fat, lecithin and sterols, which can likewise be used for producing a preparation according to the invention. In a preferred formulation of the preparation according to the invention, O/W emulsifiers can be selected from the group of plant protein hydrolysates and the derivatives thereof.

In the spirit of the present invention, furthermore substances may advantageously be present as additives, selected from the group consisting of the esters of saturated and/or unsaturated, branched and/or unbranched alkane carboxylic acids and/or alkene carboxylic acids having a chain length of 3 to 30 carbon atoms, and saturated and/or unsaturated, branched and/or unbranched alcohols having a chain length of 3 to 30 carbon atoms, and from the group consisting of esters of aromatic carboxylic acids and saturated and/or unsaturated, branched and/or unbranched alcohols having a chain length of 3 to 30 carbon atoms. Such esterols can advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethyihexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, such as jojoba oil.

The oil phase can also advantageously be selected from the group consisting of branched and unbranched hydrocarbons and hydrocarbon waxes, dialkyl ethers, from the group consisting of saturated or unsaturated, branched or unbranched alcohols, and also fatty acid triglycerides, which is to say the triglycerol esters of saturated and/or unsaturated, branched and/or unbranched alkanecarboxylic acids having a chain length of 8 to 24 carbon atoms, in particular 12 to 18 carbon atoms. The fatty acid triglycerides can advantageously be selected, for example, from the group consisting of synthetic, semisynthetic and natural oils.

The invention therefore also relates to a preparation according to the invention, comprising, for example, jojoba oil, olive oil, sunflower oil, soy bean oil, peanut oil, rapeseed oil, almond oil, palm oil, coconut oil, palm kernel oil, or the like, as the additives. Particularly preferred are vegetable oils comprising at least 6% by weight of polyunsaturated fatty acids.

In a further preferred embodiment, the preparations according to the invention comprise 1% by weight, preferably 1 to 30% by weight, particularly preferred 2 to 15% by weight, or even 5 to 10% by weight, of at least one such vegetable oil as the additive.

In particular, additionally antioxidants and/or radical scavengers can be added as auxiliary agents or additives to the preparations according to the invention. Advantageously such antioxidants are selected from the group consisting of the lipophilic systems, such as: natural and synthetic tocopherols, nordihydroguaiac acid, coniferyl benzoate, butylhydroxyanisol, butylhydroxytoluene, gallic acid ester, and different antioxidative plant extracts. Among the hydrophilic systems, particularly advantageously inorganic sulfur compounds, sodium hydrogen sulfite, cysteine or ascorbic acid are used.

The cosmetic and dermatological preparations according to the invention can furthermore comprise cosmetic auxiliary agents such as those conventionally used in such preparations, such as preservatives, bactericides, scents, antifoaming substances, dyes, pigments having a coloring effect, thickeners, surfactants, softeners, moisturizers and/or humectants, or other common constituents of a cosmetic or dermatological formulation, such as alcohols, polyols, polymers, foam stabilizers, electrolytes, organic solvents, or silicone derivatives

In a further special embodiment, the preparation according to the invention is substantially composed of naturally occurring ingredients, as mentioned above.

The invention further relates to the use of the composition according to the invention for the prophylaxis and treatment of skin diseases, in particular acne, seborrhea, atopic eczema (neurodermatitis), hirsutism, psoriasis, and dry/atopic skin of humans.

The invention further relates to a pharmaceutical drug for the treatment of skin diseases in humans, particularly for the prophylaxis and treatment of skin diseases, in particular acne, seborrhea, atopic eczema (neurodermatitis), hirsutism, psoriasis, and dry/atopic skin in humans. For these purposes, the pharmaceutical drug can be applied externally to the skin of the person.

For producing the extracts according to the invention and the combinations thereof (primary active ingredient and secondary active ingredient) from the plants, reference is made to the technical teachings, which are the subject matter of EP 1368605B1, EP 0753306B1.

Below, a few exemplary embodiments will be described, without limiting the invention to these examples.

O/W emulsion comprising Cimicifuga extract:

| | |
|---|---|
| Glycerin monostearate self-emulsifying | 3.0% |
| Sorbitan stearate | 2.0% |
| Cetyl alcohol | 3.0% |
| Lanolin alcohol | 5.0% |
| Isopropyl stearate | 3.0% |
| Vegetable oils | 7.0% |
| Silicon oils | 5.0% |
| Cimicifuga extract | 3.0% |
| Glycerin | 5.0% |
| Calendula extract | 2.5% |
| Polyacrylic acid | 0.2% |
| TriethanoLamine | 0.3% |
| Preservative | 0.4% |
| Scent | 0.3% |
| Water up to | 100.0% |

W/O emulsion comprising Cimicifuga extract:

| | |
|---|---|
| Glyceryl sorbitan isostearate | 8.0% |
| Methyl glucose dioleate | 2.0% |
| Tallow glycerides | 2.0% |
| Lanolin alcohol | 2.0% |
| Caprylic/capric glycerides | 5.0% |
| Isopropyl stearate | 8.0% |
| Silicon oil | 2.0% |
| Vegetable oils | 5.0% |
| Cimicifuga extract | 4.0% |
| Calendula extract | 3.0% |
| Glycerin | 4.0% |
| Preservative | 0.4% |
| Scent | 0.4% |
| Water up to | 100.0% |

Facial care gel comprising Cimicifuga:

| | |
|---|---|
| Carbopol | 0.8% |
| Cellulose gum | 0.1% |
| Butylene glycol | 6.0% |
| Sodium hydroxide | 0.3% |
| Cimicifuga extract | 3.5% |
| Calendula extract | 2.5% |
| Preservative | 0.4% |
| Ethyl alcohol | 8.0% |
| Scent | 0.2% |
| Water up to | 100.0% |

Facial lotion comprising Cimicifuga:

| | |
|---|---|
| Butylene glycol | 6.0% |
| Emulsifier | 2.0% |
| Ethyl alcohol | 25.0% |
| Cimicifuga extract | 4.0% |
| Calendula extract | 3.0% |
| Scent | 0.3% |
| Water up to | 100.0% |

In all examples, Cimicifuga is the primary active ingredient and can be supported in the effect by a secondary active ingredient, such as soy, red clover, hops, grape skins instead of or as part of Calendula.

1. A cosmetic or dermatological composition for topical use, comprising as the primary active ingredient 1.0 to 50% by weight Cimicifuga for the prophylaxis and treatment of skin diseases, particularly acne, seborrhea, atopic eczema (neurodermatitis), hirsutism, psoriasis and dry/atopic skin of humans, and further auxiliary agents and/or additives.

2. The cosmetic or dermatological composition for topical use according to claim 1, wherein it comprises 2.0 to 30% by weight of Cimicifuga.

3. The cosmetic or dermatological composition for topical use according to claim 1, additionally comprising extracts of soy, red clover, hops, grape skins and Calendula.

4. The cosmetic or dermatological composition for topical use according to claim 1, wherein it contains 0.1 to 15% by weight of unsaponifiable constituents of at least one further vegetable oil.

5. The cosmetic or dermatological composition for topical use according to claim 1, in the form of a cream, gel, lotion, paste, ointment, skin oil, or emulsion.

6. The cosmetic or dermatological composition for topical use according to claim 1, wherein it comprises at least one further vegetable oil, which includes at least 6% by weight of polyunsaturated fatty acids, as an additive.

7. The cosmetic or dermatological composition for topical use according to claim 1, wherein it comprises one or more additives selected from the group consisting of jojoba oil, olive oil, sunflower oil, soy bean oil, peanut oil, rapeseed oil, almond oil, palm oil, coconut oil, or palm kernel oil.

8. The cosmetic or dermatological composition for topical use according to claim 1, wherein it comprises an O/W emulsifier, particularly a plant protein hydrolysate.

9. A method for the prophylaxis and treatment of skin diseases, said method comprising:
applying to the skin of a patient suffering from a skin disease a therapeutically effective amount of a dermatological composition comprising as the primary active ingredient 1.0 to 50% by weight Cimicifuga.

10. A pharmaceutical drug comprising a dermatological composition according to claim 1 for the prophylaxis and treatment of skin diseases of humans.

11. The cosmetic or dermatological composition for topical use according to claim 1, wherein it comprises 2.5 to 20% by weight of Cimicifuga.

12. The cosmetic or dermatological composition for topical use according to claim 1, wherein it comprises 2.5 to 10% by weight of Cimicifuga.

13. The cosmetic or dermatological composition for topical use according to claim 8, wherein the O/W emulsifier is a plant protein hydrolysate.

14. The method according to claim 9, wherein said skin disease is selected from the group consisting of acne, seborrhea, atopic eczema (neurodermatitis), hirsutism, psoriasis, and dry/atopic skin of humans.

15. The pharmaceutical drug according to claim 10, wherein said drug is effective for for the prophylaxis and treatment of a condition selected from acne, seborrhea, atopic eczema (neurodermatitis), hirsutism, psoriasis, and dry/atopic skin of humans.
